(19) United States
(12) Patent Application Publication
(10) Pub. No.: US 2018/0344956 A1
(43) Pub. Date: Dec. 6, 2018

(54) Inhaler Device for Administering Powered Pharmaceutical Compositions via Inhalation

(71) Applicants: CAI GU HUANG, Shrewsbury, MA (US); JIAN CAI, Shanghai (CN); Daniel Huang, Shrewsbury, MA (US)

(72) Inventors: CAI GU HUANG, Shrewsbury, MA (US); JIAN CAI, Shanghai (CN); Daniel Huang, Shrewsbury, MA (US)

(21) Appl. No.: 15/615,114

(22) Filed: Jun. 6, 2017

(51) Int. Cl. A61M 15/00; A61K 9/00; A61K 9/14; A61K 31/46; A61M 16/14

(52) U.S. Cl. CPC A61M 15/0041; A61K 9/0075; A61K 9/145; A61K 31/46; A61M 2202/064; A61M 15/0026; A61M 15/0035; A61M 15/0086; A61M 16/14

(57) ABSTRACT

An inhaler device for administering powdered pharmaceutical composition from capsules comprises of a base, a deck, a chamber, a mouthpiece and a cap. The deck can be latched to the base and closed off, and being connected to a push button (17) through a protrusion (22) and a sliding groove (16). The chamber is for holding the capsules and can be adjusted to be lowered into the base. The mouthpiece is latch-able to the deck. The cap covers the mouthpiece in a closed position and latches it by means of a closure element. The base, the deck, the mouthpiece are connected by means of a spindle. The cap is not connected to the rest of the device parts; instead, it is held together with the rest of the inhaler device by means of the protective shell (26) with a strip pattern (27) or a linking belt (29). Finally, there is a capsule-piercing unit with at least one piercing pin for piercing the capsule.

Benefits of U.S. Provisional Application No. 62/493,371, filed Jul. 1, 2016 and No. 62/498,588, filed Jan. 3, 2017 is hereby claimed.

### BACKGROUND

This invention refers to an inhaler device for administering powdered pharmaceutical composition from capsules that are held by a chamber of the device. After the capsule has been put into the chamber, the user can employ a capsule-piercing unit that includes at least one piercing pin to pierce the capsule. After the piercing process, the pharmaceutical composition powder can be released from the capsule and inhaled by the patient.

EP 0703800 B1 or EP 0911047 A1 and U.S. Pat. No. 7,694,676 are examples for this type of inhaler device. The inhaler devices in these disclosures have a base and a cap which are both dish-shaped like.

In the current invention, these two parts complement each other and can be flipped open or closed for use through a protective shell (26) with a strip pattern (27) or a linking belt (29). The base of the device, including the mouthpiece and a deck, operates through a joint spindle (6). The mouthpiece is also capable of being flipped open or closed. The deck is below the mouthpiece. The deck can be latched to the base and closed off, and is connected to a push button (17) through a protrusion (22) and a sliding grove (16). After the device has been flipped open, the user can load a powdered capsule in the chamber, close the chamber and the mouthpiece into the deck, and push the button (17) to utilize a spring loaded capsule-piercing unit which moves laterally from the base to pierce the capsule. The user then inhales the powdered pharmaceutical composition into his airway through the mouthpiece.

The aim of this invention is to enhance the ease of handling the inhalation device.

### SUMMARY OF THE INVENTION

According to a first embodiment, the current invention is directed to an inhaler device for administering powdered pharmaceutical compositions from capsules. This device is comprised of a base, a deck, a chamber, a mouthpiece and a cap, wherein the deck can be latched to the base and closed off. The deck is connected to a push button (17) through a protrusion (22) and a sliding groove (16), wherein the push button (17) and spring fixture (18) are not hinged together and kept as two separated parts. The base, the deck, the mouthpiece and the cap are all hinged together by a single joint spindle (6) (refer to FIG. 1).

According to a second embodiment, the current invention is directed to an inhaler device for administering powdered pharmaceutical compositions from capsules. This device is comprised of a base, a deck, a chamber, a mouthpiece and a cap, wherein the deck can be latched to the base and closed off. The deck is connected to a push button (17) through a protrusion (22) and a sliding groove (16), wherein the push button (17) and spring fixture (18) are not hinged together and kept as two separated parts. The cap is not hinged together by a single spindle joint with the rest of the device and instead it is held together with the rest of the inhaler device by means of a bugle on the inside of the cap along with the corresponding dent on the mouthpiece, and by means of the protective shell (26) with a strip pattern (27) (refer to FIG. 5 and FIG. 6).

According to a third embodiment, the current invention is directed to an inhaler device for administering powdered pharmaceutical compositions from capsules. This device is comprised of a base, a deck, a chamber, a mouthpiece and a cap, wherein the deck can be latched to the base and closed off. The deck is connected to a push button (17) through a protrusion (22) and a sliding groove (16), wherein the push button (17) and spring fixture (18) are not hinged together and kept as two separated parts. The cap in the device is connected to the rest of the device parts by means of a linking belt (29) which attaches the cap and the push button on both sides (refer to FIG. 7).

The mouthpiece in the device has a clench section in order to ensure fast and solid opening. The clench section and the sucking section are located on different regions of the mouthpiece and can be discerned unambiguously due to the shape and appearance of these sections and the mouthpiece. Therefore, the patient can open the mouthpiece without contaminating the sucking section; this is especially critical since the sucking section is in contact with the mouth during the inhalation process.

According to the invention, the significance of the special opening pattern for the cap and the clench section on the mouthpiece is to ensure easier handling of the inhaler device for patients. This is vital for time-sensitive situations such as asthma attacks, where patients may experience difficulty in using the device properly.

In a preferred embodiment, besides the spring element between the capsule-piercing unit and the capsule holder, there may be at least one other spring element applied between the deck and the base to aid the opening process. This additional spring element can allow the cap and/or the mouthpiece to spring open.

In a preferred embodiment, the push button of the capsule-piercing unit is mounted on the deck through the sliding groove (16), and the spring fixture 18 and its associated parts of the capsule-piercing unit is mounted on the chamber. Therefore, the capsule-piercing unit abuts the deck and the chamber, and the capsule-piercing unit can slide along the deck through the sliding groove (16) from the resting position to the functional position guided by arms.

In a preferred embodiment, the capsule-piercing unit is loaded with a spring element. The deformation of the spring element in the function position can ensure the spring's return to its resting position; this allows for more rapid re-use of the device's functional capabilities.

Advantageously, the capsule-piercing unit consists of a push button, spring fixture, piercing pin and spring element. Mounted on the chamber, the spring fixture (18) has arms and can lead the piercing elements and the spring to move between the resting position and the functional position. The arms have end bulges to ensure the arm can be entrapped on each side of the capsule chamber to prevent the spring fixture from completely departing from the chamber at the resting position.

In a preferred embodiment, the push button of the capsule-piercing unit has rifled surfaces so that it can help provide optimal grip during use. Advantageously, the push button has an inclining dent on its upside so that it forms a sliding surface for the closure part of the cap so it can detach the cap from the base when the capsule-piercing unit is pushed.

Advantageously, the deck connected to the base can be easily removed from the base for cleaning and disinfecting purposes. The connection between the deck and the base is established by the retaining buckles on the deck.

In all the embodiments, it is possible that the inhaler device is designed in such a way so that the capsule-piercing unit can be detached from the base, together with the deck.

Additional advantages, aspects and features of the present invention will be apparent from the description and drawings referenced below.

### BRIEF DESCRIPTION OF THE DRAWINGS

The drawings of the inhaler device are described and presented in FIG. 1, FIG. 2, FIG. 3, FIG. 4, FIG. 5, FIG. 6, FIG. 7 and FIG. 8, respectively.

FIG. 1 shows an open state profile view of the inhaler device;

FIG. 2 shows an internal view of the cap and mouthpiece;

FIG. 3 shows a top-down view of the deck and base;

FIG. 4 shows a profile view of the capsule-piercing unit;

FIG. 5 shows a profile of the preferred embodiment of a separated cap (closed cap position);

FIG. 6 shows a profile of the preferred embodiment of a separated cap (open cap position);

FIG. 7 shows a profile of the preferred embodiment with the attached linker;

FIG. 8 shows an enlarged view of the metal mesh

### DETAILED DESCRIPTION OF THE INVENTION

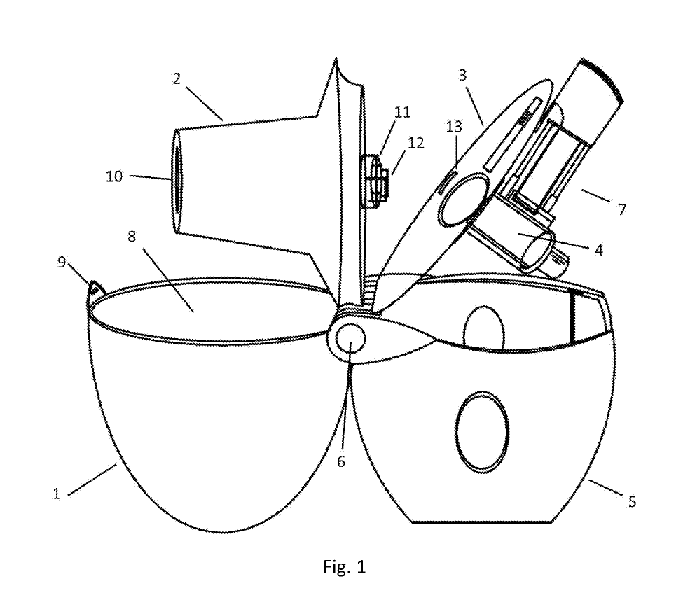

According to a first embodiment, the current invention is directed to an inhaler device for administering powdered pharmaceutical compositions from capsules. This device is comprised of a base, a deck, a chamber, a mouthpiece and a cap, wherein the deck can be latched to the base and closed off. The deck is connected to a push button (17) through a protrusion (22) and a sliding groove (16), wherein the push button (17) and spring fixture (18) are not hinged together with two separated parts. The base, the deck, the mouthpiece and the cap are connected together by a single joint spindle (6) (refer to FIG. 1).

FIG. 1 shows the open state view of the inhaler device of this embodiment. The inhaler device consists of a cap 1, a mouthpiece 2, a deck 3, a chamber 4, a base 5, a single joint spindle 6 and a capsule-piercing unit 7. The cap 1 has a cavity 8 for protecting the mouthpiece 2. The cap 1 has a buckle 9 near the open position to ensure effective closure of the inhaler device. The mouthpiece 2 has an inhalable passage 10 coaxially disposed. The inhalable passage 10 is approximately cylindrical whereas the inhalable passage 10 expands at both ends. The inhalable passage 10 is embedded with a metal mesh 11, and the metal mesh 11 protrudes slightly outward. Near the metal mesh 11, pair of fasteners work with a pair of slots 13 on the deck 3 to ensure a tight connection between the inhalable passage 10 and the chamber 4. According to FIG. 1, the chamber 4 is installed vertically under the deck 3. When the mouthpiece 2 is apart from the chamber 4, inhalable powdered capsule can be vertically located in the chamber 4. The deck 3, which is roughly elliptical, has a pair of buckles 14 to ensure a tight connection between the deck 3 and the base 5. Two gaps 15 between the deck 3 and the base 5 are left as air flow entrances. The two gaps 15 are symmetrical with each other and their shape is long and narrow. There are two transparent windows (28) on both sides of the base (5). The transparent windows (28) are fashioned with an elliptical shape. The transparent windows can be used by patients to observe the chamber (4) to determine if drug delivery is successful. In consideration of potential visual impairments (particularly in elderly patients), the transparent windows could be made with convex lenses for clearer observation of capsule usage in the chamber (4). Alternatively, the transparent windows (28) could be constructed with concave lenses and thereby the internal condition of the device could be better observed. Ideally, one side of the base (5) would contain a concave lens and the other side a convex lens.

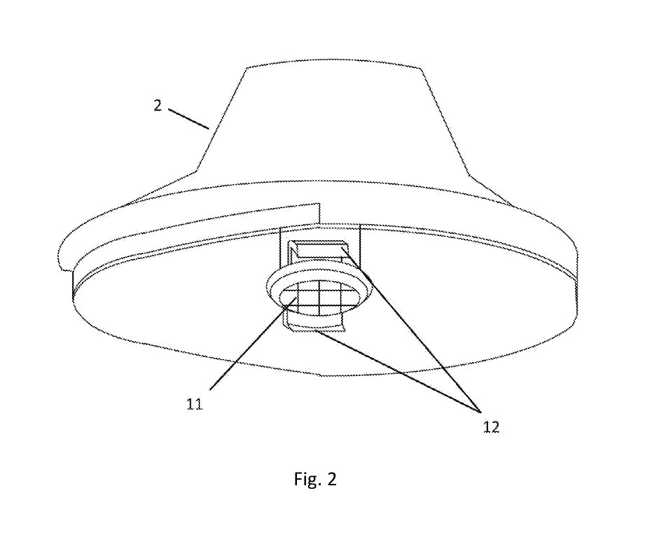

FIG. 2 shows the inhalable passage 10, the metal mesh 11 and the pair of fasteners 12 from another viewing angle.

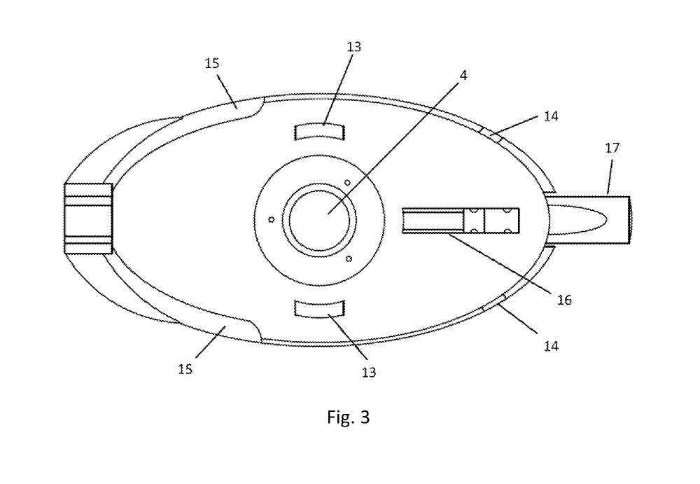

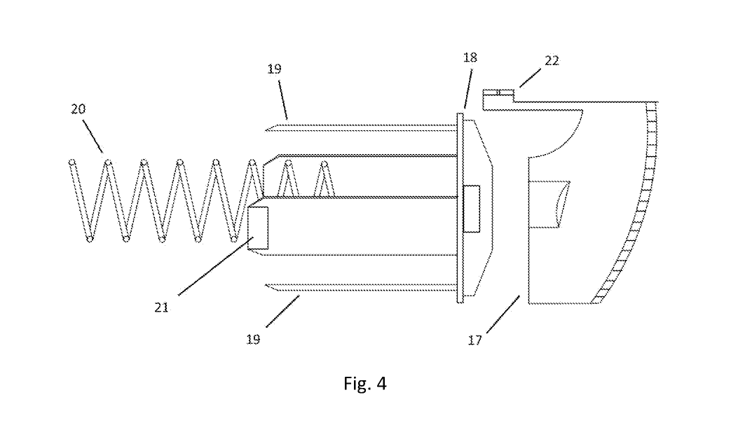

FIG. 3 shows that the deck 3 includes a pair of slots 13 and a sliding groove 16. The sliding groove is used to control the position and sliding direction of a capsule-piercing unit 7. In particular, the capsule-piercing unit 7 consists of a push button 17, spring fixture 18, two piercing elements 19 and a spring 20. The push button 17 has a protrusion 22, which is entrapped in the sliding groove 16 to prevent the push button 17 from dropping and would not increase the operational difficulty of piercing capsules. The spring fixture 18 has two arms 23. Each arm has at least one bulge 21 to ensure the arm could be entrapped in the each side of the chamber 4 so that it can prevent the spring fixture 18 from completely moving away from the chamber 4. Mounted outward the chamber 4, the spring fixture 18 can encompass the spring 20, and can also lead the piercing elements 19 and the spring 20 to move according to a specified direction and range. Inside the spring fixture 18, the piercing element 19 is a sharpened pin. The tip of piercing element 19 can be sharpened to a certain angle or be symmetrically sharpened to ensure effective capsule piercing. After the piercing action, the stressed spring 20 can ensure that the capsule-piercing unit 7 is retracted. The capsule-piercing unit 7 is designed to have two plastic parts (the push button 17 and the spring fixture 18) so that the stability and precision of it can be enhanced and that it should have more advantages in mass production. For instance, this design reduces mould complexity and increases production efficiency. FIG. 4 shows the capsule-piercing unit 7.

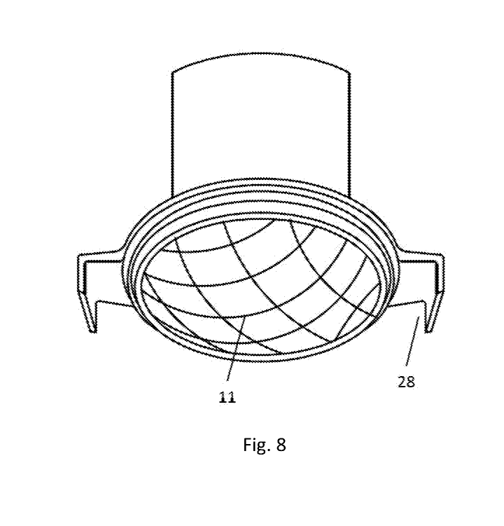

FIG. 8 is an enlarged view of the metal mesh. The grid interval of metal mesh (11) is between 0.5 mm and 2 mm. The metal mesh (11) has a certain hemi-ellipsoidal arc. The arc of metal mesh (11) is toward the direction of capsule chamber. When the patients inhale, the arc of metal mesh (11) can aid the vibration of the capsule in the chamber, thereby increasing the effectiveness of drug administration.

The cap 1, the mouthpiece 2, the deck 3 and the base 5 are connected through the spindle 6 so that these parts cannot be lost by the user. When using the inhaler device, first hold the base 5 steadily; next, flip open the cap 1 and the mouthpiece 2. Put the capsule into the chamber 4, then connect the mouthpiece 2 and deck 3 tightly through the pair of fasteners 12 and the pair of slots 13. Push the button 17 until the capsule is pierced successfully. Next, exhale completely and insert the mouthpiece 2 into the mouth and ensure the mouthpiece 2 is tightly covered; inhale quickly. Once the user inhales, the surrounding air will enter into the base 5 through the gaps 15 and then enter into the chamber 4. Once in the chamber 4, the air flow will have a faster speed and more powerful turbulence, which will help deliver the inhalable powder out of the capsule and into the user's mouth through the metal mesh 11 and the inhalable passage 10. Herein the metal mesh 11 can prevent the capsule from being inhaled into the inhalable passage 10 and into the user's mouth.

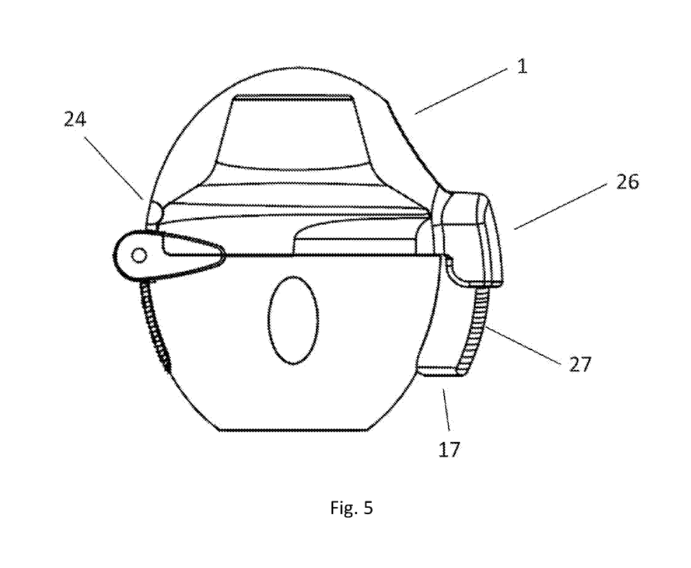

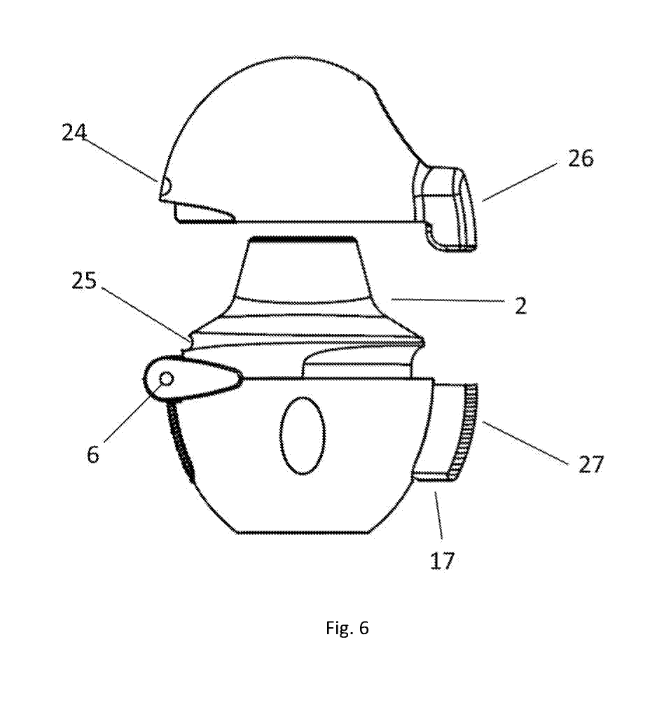

According to a second embodiment, the current invention is directed to an inhaler device for administering powdered pharmaceutical compositions from capsules. This device is comprised of a base, a deck, a chamber, a mouthpiece and a cap, wherein the deck can be latched to the base and closed off. The deck is connected to a push button (17) through a protrusion (22) and a sliding groove (16), wherein the push button (17) and spring fixture (18) are not hinged together with two separated parts. The cap is not hinged together by a single spindle joint with the rest of the device and instead it is held together with the rest of the inhaler device by means of a bugle on the inside of the cap along with the corresponding dent on the mouthpiece, and by means of the protective shell (26) with a strip pattern (27) (refer to FIG. 5 and FIG. 6).

FIGS. 5 and FIG. 6 show the inhaler device of this embodiment. In this device, the cap (1) is not connected to the rest of the device and can be completely separated. Near the spindle (6), there is a bulge (24) on the inside of cap (1) along with a corresponding dent (25) on the mouthpiece (2). Near the push button (17), there is a push button protective shell (26). Under the closed cap state, the protective shell (26) could potentially reduce the unintentional actuation of the push button (17) from users. Moreover, under the closed cap state, the push button (17) will exert a certain pressure force from the spring (20) to the protective shell (26), thereby contributing to the tight binding between the bulge (24) and the dent (25) while also contributing to the fastening between the strip pattern (27) on the push button (17) and the strip pattern (not displayed on the figures) on the inside of the protective shell (26). When the cap (1) is closed correctly, this will largely reduce unwanted and accidental opening of the device, thereby avoiding contamination of the mouthpiece (2) and other components. The correct way to open the cap is (1): push the button (17), then pull the cap (1), then loosen the push button (17). The correct way to close the cap (1) is as follows: push the button (17), close the cap (1), loosen the button (17), and the cap (1) will close.

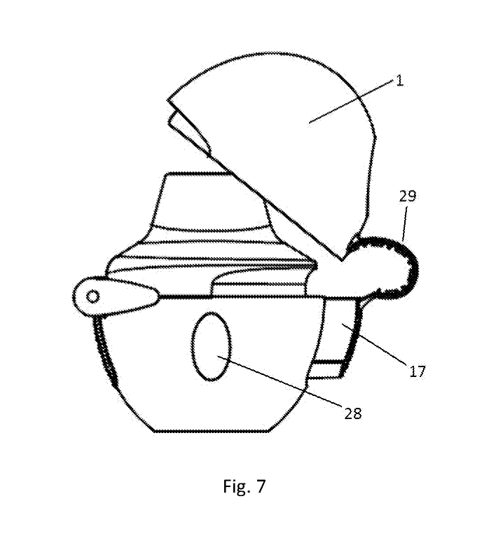

According to a third embodiment, the current invention is directed to an inhaler device for administering powdered pharmaceutical compositions from capsules. This device is comprised of a base, a deck, a chamber, a mouthpiece and a cap, wherein the deck can be latched to the base and closed off. The deck is connected to a push button (17) through a protrusion (22) and a sliding groove (16), wherein the push button (17) and spring fixture (18) are not hinged together with two separated parts. The cap in the device is connected to the rest of the device parts by means of a linking belt (29) which attaches the cap and the push button on both sides (refer to FIG. 7).

FIG. 7 shows a profile view of the embodiment with a linking belt. This embodiment uses an elastic linking belt to connect the cap with the rest of the device and the push button in order to avoid loss of the cap.

According to another aspect of the invention, the pharmaceutical composition used for inhalation may be all kinds of suitable powdered pharmaceuticals comprised of a pharmaceutical active substance or a combination of a pharmaceutical active substance and a carrier.

According to a further aspect of invention, the pharmaceutical active ingredients listed below may be used in the device on their own or in combination. Preferably the pharmacologically active substances are selected from the betamimetics, anticholinergics, corticosteroids, PDE4-inhibitors, LTD4-antagonists, EGFR-inhibitors, dopamine agonists, H1-antihistamines, PAF4-antagonists and PI3-kinase inhibitors. Moreover, double or triple combinations of above compounds may be combined and used in the device.

For example, a betamimetic may be combined with an anticholinergic, corticosteroid, PDE4-inhibitor, EGFR-inhibitor or LTD4-antagonist; an anticholinergic may be combined with a betamimetic, corticosteroid, PDE4-inhibitor, EGFR-inhibitor 60 or LTD4-antagonist; a corticosteroid may be combined with a PDE4-inhibitor, EGFR-inhibitor or LTD4-antagonist; a PDE4-inhibitor may be combined with an EGFR-inhibitor or LTD4-antagonist; an EGFR-inhibitor may be combined with an LTD4-antagonist.

According to a further aspect of the invention, the pharmaceutical active substances used as betamimetics are preferably compounds selected from among vilanterol, olodaterol, Indacaterol, albuterol, arformoterol, bam-buterol, bitolterol, broxaterol, carbuterol, clenbuterol, fenotrol, formoterol, metaprotereol, hexoprenaline, ibuterol, isoetharine, isoprenaline, levosalbutamol, mabuterol, meluadrine, metaproterenol, orciprenaline, pirbuterol, procaterol, reproterol, rimiterol, ritodrine, salmefamol, salmeterol, soterenol, sul-phonterol, terbutaline, tiaramide, tolubuterol, zinterol, CHF-1035, HOKU-81, KUL-1248 and 3-(4-{6-[2-hydroxy-2-(4-hydroxy-3-hydroxymethyl-phenyl)-ethyamino]-hexyloxy}-butyl)-benzyl-sulphonamide,

5-[2-(5,6-diethylindan-2-ylamino)-1-hydroxy-ethyl]-8-hydroxy-1H-quinolin-2-one,
4-hydroxy-7-[2-{[2-{[3-(2-phenylethoxy)propyl]sulphonyl}ethyl]-amino}ethyl]-2(3)-benzothiazolone,
1-(2-fluoro-4-hydroxyphenyl)-2-[4-(1-benzimidazolyl)-2-methyl-2-butylamino]ethanol,
1-[3-(4-methoxybenzyl-amino)-4-hydroxyphenyl]-2-[4-(1-benzimidazolyl)-2-methyl-2-butylamino]ethanol,
1-[2H-5-hydroxy-3-oxo-4H-1,4-benzoxazin-8-yl]-2-[3-(4-N,N-dimethylaminophenyl)-2-methyl-2-propylamino]ethanol,
1-[2H-5-hydroxy-3-oxo-4H-1,4-benzoxazin-8-yl]-2-[3-(4-methoxyphenyl)-2-methyl-2-propylamino]ethanol,
1-[2H-5-hydroxy-3-oxo-4H-1,4-benzoxazin-8-yl]-2-[3-(4-n-butyloxyphenyl)-2-methyl-2-propylamino]ethanol,
1-[2H-5-hydroxy-3-oxo-4H-1,4-benzoxazin-8-yl]-2-{4-[3-(4-methoxyphenyl)-1,2,4-triazol-3-yl]-2-methyl-2-butylaminojethanol,
5-hydroxy-8-(1-hydroxy-2-isopropylaminobutyl)-2H-1,4-benzoxazin-3-(4H)-one,
1-(4-amino-3-chloro-5-trifluoromethylphenyl)-2-tert.-buty-lamino)ethanol,
6-hydroxy-8-{1-hydroxy-2-[2-(4-methoxy-phenyl)-1,1-dimethyl-ethylamino]-ethyl}-4H-benzo[1,4]oxazin-3-one,
6-hydroxy-8-{1-hydroxy-2-[2-(ethyl-4-phenoxy-acetate)-1,1-dimethyl-ethylamino]-ethyl}-4H-benzo[1,4]oxazin-3-one,
6-hydroxy-8-{1-hydroxy-2-[2-(4-phenoxy-aceticacid)-1,1-dimethyl-ethylamino]-ethyl}-4H-benzo[1,4]oxazin-3-one,
8-{2-[1,1-dimethyl-2-(2,4,6-trimethylphenyl)-ethylamino]-1-hydroxy-ethyl}-6-hydroxy-4H-benzo[1,4]oxazin-3-one,
6-hydroxy-8-{1-hydroxy-2-[2-(4-hydroxy-phenyl)-1,1-dimethyl-ethylamino]-ethyl}-4H-benzo[1,4]oxazin-3-one,
6-hydroxy-8-{1-hydroxy-2-[2-(4-isopropyl-phenyl)-1,1-dimethyl-ethylamino]-ethyl}-4H-benzo[1,4]oxazin-3-one,
8-{2-[2-(4-ethyl-phenyl)-1,1-dimethyl-ethylamino]-1-hydroxy-ethyl}-6-hydroxy-4H-benzo[1,4]oxazin-3-one,
8-{2-[2-(4-ethoxy-phenyl)-1,1-dimethyl-ethylamino]-1-hydroxy-ethyl}-6-hydroxy-4H-benzo[1,4]oxazin-3-one,
4-(4-{2-[2-hydroxy-2-(6-hydroxy-3-oxo-3,4-dihydro-2H-benzo[1,4]oxazin-8-yl)-ethylamino]-2-methyl-propyl}-phenoxy)-butyric acid,
8-{2-[2-(3,4-difluoro-phenyl)-1,1-dimethyl-ethylamino]-1-hydroxy-ethyl}-6-hydroxy-4H-benzo[1,4]oxazin-3-one,
1-(4-ethoxy-carbonylamino-3-cyano-5-fluorophenyl)-2-(tert-butylamino)ethanol,
2-hydroxy-5-(1-hydroxy-2-{2-[4-(2-hydroxy-2-phenyl-ethylamino)-phenyl]-ethylamino}-ethyl)-benzaldehyde,
N-[2-hydroxy-5-(1-hydroxy-2-{2-[4-(2-hydroxy-2-phenyl-ethylamino)-phenyl]-ethylamino}-ethyl)-phenyl]-formamide,
8-hydroxy-5-(1-hydroxy-2-{2-[4-(6-methoxy-biphenyl-3-ylamino)-phenyl]-ethylamino}-ethyl)-1H-quinolin-2-one,
8-hydroxy-5-[1-hydroxy-2-(6-phenethylamino-hexy-lamino)-ethyl]-1H-quinolin-2-one,
5-[2-(2-{4-[4-(2-amino-2-methyl-propoxy)-phenylamino]-phenyl}-ethylamino)-1-hydroxy-ethyl]-8-hydroxy-1H-quinolin-2-one,
[3-(4-{6-[2-hydroxy-2-(4-hydroxy-3-hydroxymethyl-phenyl)-ethylamino]-hexyloxy}-butyl)-5-methyl-phenyl]-urea,
4-(2-{6-[2-(2,6-dichloro-benzyloxy)-ethoxy]-hexylamino}-1-hydroxy-ethyl)-2-hydroxymethyl-phenol,
3-(4-{6-[2-hydroxy-2-(4-hydroxy-3-hydroxymethyl-phenyl)-ethylamino]-hexyloxy}-butyl)-benzylsulphonamide,
3-(3-{7-[2-hydroxy-2-(4-hydroxy-3-hydroxymethyl-phenyl)-ethylamino]-heptyloxy}-propyl)-benzylsulphonamide,
4-(2-{6-[4-(3-cyclopentanesulphonyl-phenyl)-butoxy]-hexylamino}-1-hydroxy-ethyl)-2-hydroxymethyl-phenol,
N-adamantan-2-yl-2-(3-{2-[2-hydroxy-2-(4-hydroxy-3-hy-droxymethyl-phenyl)-ethylamino]-propyl}-phenyl)-acetamide,
optionally in the form of the racemates, enantiomers, diastereomers thereof and optionally in the form of the pharmacologically acceptable acid addition salts, solvates or hydrates thereof. According to the invention, the acid addition salts of the betamimetics are preferentially selected from among the hydrochloride, hydrobromide, hydriodide, hydrosulphate, hydrophosphate, hydromethanesulphonate, hydronitrate, hydromaleate, hydroacetate, hydrocitrate, hydrofumarate, hydrotartrate, hydroxalate, hydrosuccinate, hydrobenzoate and hydro-p-toluenesulphonate.

According to another aspect of the invention, the anticholinergics used are preferably compounds selected from among aclidinium salts, preferably the bromide salts, umeclidinium salts, tiotropium salts, preferably the bromide salt and monohydrate, oxitropium salts, flutropium salts, ipratropium salts, glycopyrronium salts, trospium salts, preferably the chloride salt, tolterodine. In the above-mentioned salts the cations are the pharmacologically active constituents. As anions the above-mentioned salts may preferably contain the chloride, bromide, iodide, sulphate, phosphate, methanesulphonate, nitrate, maleate, acetate, citrate, fumarate, tartrate, oxalate, succinate, benzoate or p-toluenesulphonate, while chloride, bromide, iodide, sulphate, methanesulphonate or p-toluene-sulphonate are preferred as counter-ions. Of all the salts the chlorides, bromides, iodides and methanesulphonates are particularly preferred.

Other preferred anticholinergics are selected from among the salts of formula AC-1

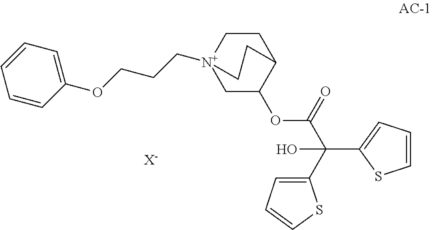

wherein X− denotes an anion with a single negative charge, preferably an anion selected from among the fluoride, chloride, bromide, iodide, sulphate, phosphate, methanesulphonate, nitrate, maleate, acetate, citrate, fumarate, tartrate, oxalate, succinate, benzoate and p-toluenesulphonate, preferably an anion with a single negative charge, particularly preferably an anion selected from among the fluoride, chloride, bromide, methanesulphonate and p-toluenesulphonate, particularly preferably bromide, optionally in the form of the racemates, enantiomers or hydrates thereof. Of particular importance are those pharmaceutical combinations which contain the enantiomers of formula AC-1-en

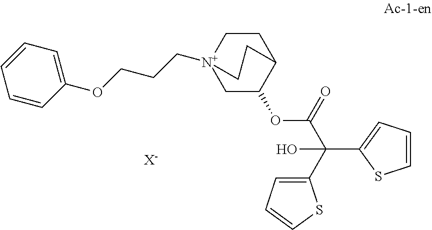

wherein X− may have the above-mentioned meanings. Other preferred anticholinergics are selected from the salts of formula AC-2

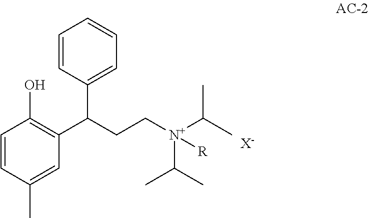

wherein R denotes either methyl or ethyl and wherein X− may have the above-mentioned meanings. In an alternative embodiment the compound of formula AC-2 may also be present in the form of the free base AC-2-base.

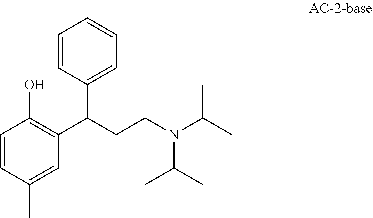 [SMILES: Cc1ccc(O)c(C(CCN(C(C)C)C(C)C)c2ccccc2)c1]

Other specified compounds are: tropenol 2,2-diphenylpropionate methobromide,

scopine 2,2-diphenylpropionate methobromide,
scopine 2-fluoro-2,2-diphenylacetate methobromide,
tropenol 2-fluoro-2,2-diphenylacetate methobromide;
tropenol 3,3,4,4,-tetrafluorobenzilate methobromide,
scopine 3,3′,4,4′-tetrafluorobenzilate methobromide,
tropenol 4,4′-difluorobenzilate methobromide,
scopine 4,4′-difluorobenzilate methobromide,
tropenol 3,3′-difluorobenzilate methobromide,
scopine 3,3,-difluorobenzilate methobromide,
tropenol 9-hydroxy-fluorene-9-carboxylate methobromide,
tropenol 9-fluoro-fluorene-9-carboxylate methobromide,
scopine 9-hydroxy-fluorene-9-carboxylate methobromide,
scopine 9-fluoro-fluorene-9-carboxylate methobromide,
tropenol 9-methyl-fluorene-9-carboxylate methobromide,
scopine 9-methyl-fluorene-9-carboxylate methobromide,
cyclopropyltropine benzilate methobromide,
cyclopropyltropine 2,2-diphenylpropionate methobromide,
cyclopropyltropine 9-hydroxy-xanthene-9-carboxylate methobromide,
cyclopropyltropine 9-methyl-fluorene-9-carboxylate methobromide,
cyclopropyltropine 9-methyl-xanthene-9-carboxylate methobromide,
cyclopropyltropine 9-hydroxy-fluorene-9-carboxylate methobromide;
cyclopropyltropine methyl 4,4′-difluorobenzilate methobro-mide.
tropenol 9-hydroxy-xanthene-9-carboxylate methobromide,
scopine 9-hydroxy-xanthene-9-carboxylate methobromide,
tropenol 9-methyl-xanthene-9-carboxylate methobromide,
scopine 9-methyl-xanthene-9-carboxylate methobromide,
tropenol 9-ethyl-xanthene-9-carboxylate methobromide,
tropenol 9-difluoromethyl-xanthene-9-carboxylate methobromide,
and scopine 9-hydroxymethyl-xanthene-9-carboxylate methobromide.

The above-mentioned compounds may also be used as salts within the scope of the present invention, wherein instead of the methobromide, the salts metho-X are used, wherein X may have the meanings given hereinbefore for X⁻.

According to a further aspect of the invention, corticosteroids are preferably selected from among beclomethasone, betamethasone, budesonide, butixocort, ciclesonide, deflazacort, dexamethasone, etiprednol, flunisolide, fluticasone, fluticasone furoate, loteprednol, mometasone, prednisolone, prednisone, rofleponide, triamcinolone, RPR-106541, NS-126, ST-26 and

(S)-fluoromethyl 6,9-difluoro-17-[(2-furanylcarbonyl)oxy]-11-hydroxy-16-methyl-3-oxo-androsta-1,4-diene-17-carbothionate,
(S)-(2-oxo-tetrahydro-furan-3S-yl)6,9-difluoro-1,11-hydroxy-16-methyl-3-oxo-17-propionyloxy-androsta-1,4-diene-17-carbothionate,
cyanomethyl6a,9a-difluoro-1-1 p-hydroxy-16a-methyl-3-oxo-17a-(2,2,3,3-tertamethylcyclopropylcarbonyl)oxy-androsta-1,4-diene-17p-carboxylate,
optionally in the form of the racemates, enantiomers or diastereomers thereof and optionally in the form of the salts and derivatives thereof, the solvates and/or hydrates thereof. Any reference to steroids includes a reference to any salts or derivatives, hydrates or solvates thereof which may exist. Examples of possible salts and derivatives of the steroids may be: alkali metal salts, such as for example sodium or potassium salts, sulphobenzoates, phosphates, isonicotinates, acetates, dichloroacetates, propionates, dihydrogen phosphates, palmitates, pivalates or furoates.

PDE4-inhibitors are preferably compounds selected from among enprofyllin, theophyllin, roflumilast, ariflo (cilomilast), tofimilast, pumafentrin, lirimilast, 60 arofyllin, atizoram, D-4418, Bay-198004, BY343, CP-325.366, D-4396 (Sch-351591), AWD-12-281 (GW-842470), NCS-613, CDP-840, D-4418, PD-168787, T-440, T-2585, V-11294A, Cl-1018, CDC-801, CDC-3052, D-22888, YM-58997, Z-15370 and N-(3,5-dichloro-1-oxo-pyridin-4-yl)-4-difluoromethoxy-3-cyclopropylmethoxybenzamide,

(−)p-[(4aR*,10bS*)-9-ethoxy-1,2,3,4,4a,10b-hexahydro-8-methoxy-2-methylbenzo[s][1,6]naphthyridin-6-yl]-N,N-diisopropylbenzamide,
(R)-(+)-1-(4-bromobenzyl)-4-[(3-cyclopentyloxy)-4-meth-oxyphenyl]-2-pyrrolidone
3-(cyclopentyloxy-4-methoxyphenyl)-1-(4-N,-[N-2-cyano-S-methyl-isothioureido]benzyl)-2-pyrrolidone,
cis[4-cyano-4-(3-cyclopentyloxy-4-methoxyphenyl)cyclohexane-1-carboxylie acid]2-carbomethoxy-4-cyano-4-(3-cyclopropylmethoxy-4-dif-luoromethoxy- phenyl)cyclohexan-1-one,
cis[4-cyano-4-(3-cyclopropylmethoxy-4-difluoromethox-yphenyl)cyclohexan-1-ol],
(R)-(+)-ethyl[4-(3-cyclopentyloxy-4-methoxyphenyl)pyrrolidin-2-ylidene]acetate,
(S)-(−)-ethyl[4-(3-cyclopentyloxy-4-methoxyphenyl)pyrrolidin-2-ylidene]acetate,
9-cyclopentyl-5,6-dihydro-7-ethyl-3-(2-thienyl)-9H-pyrzolo[3,4-c]-1,2,4-triazolo[4,3-a]pyridine,
9-cyclopentyl-5,6-dihydro-7-ethyl-3-(tert-butyl)-9H-pyrazolo[3,4-c]-1,2,4-triazolo[4,3-a]pyridine,
optionally in the form of the racemates, enantiomers or dias-tereomers thereof and optionally in the form of the pharmacologically acceptable acid addition salts thereof, the solvates and/or hydrates thereof. According to the invention, the acid addition salts of the betamimetics are preferably selected from among the hydrochloride, hydrobromide, hydriodide, hydrosulphate, hydrophosphate, hydromethanesulphonate, hydronitrate, hydromaleate, hydroacetate, hydrocitrate, hydrofumarate, hydrotartrate, hydroxalate, hydrosuccinate, hydrobenzoate and hydro-p-toluenesulphonate.

The LTD4-antagonists are preferably selected from among zileuton, montelukast, pranlukast, zafirlukast, MCC-847 (ZD-3523), MN-001, MEN-91507 (LM-1507), VUF-5078, VUF-K-8707, L-733321 and

1-(((R)-(3-(2-(6,7-difluoro-2-quinolinyl)ethenyl)phenyl)-3-(2-(2-hydroxy-2-propyl)phenyl)thio)methylyclopropane-acetic acid,
1-(((1R)-3(3-(2-(2,3-dichlorothieno[3,2-b]pyridin-5-yl)-(E)-ethenyl)phenyl)-3-(2-(1-hydroxy-1- methylethyl)phenyl)propyl)thio)methyl)cyclopropaneacetic acid,
[2-[[2-(4-tert-butyl-2-thiazolyl)-5-benzofuranyl]oxymethyl]phenyljacetic acid,
optionally in the form of the racemates, enantiomers or dias-tereomers thereof and optionally in the form of the pharmacologically acceptable acid addition salts, solvates and/or hydrates thereof. According to the invention the acid addition salts of the betamimetics are preferably selected from among the hydrochloride, hydrobromide, hydroiodide, hydrosulphate, hydrophosphate, hydromethanesulphonate, hydronitrate, hydromaleate, hydroacetate, hydrocitrate, hydrofuma-rate, hydrotartrate, hydroxalate, hydrosuccinate, hydrobenzoate and hydro-p-toluenesulphonate. By salts or derivatives which the LTD4-antagonists may optionally be capable of forming are meant, for example: alkali metal salts, such as for example sodium or potassium salts, alkaline earth metal salts, sulphobenzoates, phosphates, isonicotinates, acetates, propionates, dihydrogen phosphates, palmitates, pivalates or furoates.

EGFR-inhibitors are preferably selected from among necitumumab, cetuximab, trastuzumab, ABX-EGF, Mab ICR-62 and

4-[(3-chloro-4-fluorophenyl)amino]-6-{[4-(morpholin-4-yl)-1-oxo-2-buten-1-yl]-amino}-7-cyclopropylmethoxy-quinazoline,
4-[(3-chloro-4-fluorophenyl)amino]-6-{[4-(N,N-diethylamino)-1-oxo-2-buten-1-1]amino}-7-cyclopropyl-methoxy-quinazoline,
4-[(3-chloro-4-fluorophenyl)amino]-6-{[4-(N,N-dimethy-lamino)-1-oxo-2-buten-1-yl]amino}-7-cyclopropyl-methoxy-quinazoline,
4-[(R)-(1-phenyl-ethyl)amino]-6-{[4-(morpholin-4-yl)-1-oxo-2-buten-1-yl]amino}-7-cyclopentyloxy-quinazoline,
4-[(3-chloro-4-fluoro-phenyl)amino]-6-{[4-((R)-6-methyl-2-oxo-morpholin-4-yl)-1-oxo-2-buten-1-yl]amino}-7-cy-clopropylmethoxy-quinazoline,
4-[(3-chloro-4-fluoro-phenyl)amino]-6-{[4-((R)-6-methyl-2-oxo-morpholin-4-yl)-1-oxo-2-buten-1-yl]amino}-7-[(S)-(tetrahydrofuran-3- yl)oxy]-quinazoline,
4-[(3-chloro-4-fluoro-phenyl)amino]-6-{[4-((R)-2-meth-oxymethyl-6-oxo-morpholin-4-yl)-1-oxo-2-buten-1-yl]amino}-7-cyclopropylmethoxy- quinazoline,
4-[(3-chloro-4-fluoro-phenyl)amino]-6-[2-((S)-6-methyl-2-oxo-morpholin-4-yl)-ethoxy]-7-methoxy-quinazoline,
4-[(3-chloro-4-fluorophenyl)amino]-6-({4-[N-(2-methoxy-ethyl)-N-methyl-amino]-1-oxo-2-buten-1-yl}amino)-7-cyclopropylmethoxy-quinazoline,
4-[(3-chloro-4-fluorophenyl)amino]-6-{[4-(N,N-dimethy-lamino)-1-oxo-2-buten-1-yl]amino}-7-cyclopentyloxy-quinazoline,
4-[(R)-(1-phenyl-ethyl)amino]-6-{[4-(N,N-to-(2-methoxy-ethyl)-amino)-1-oxo-2-buten-1-yl]amino}-7-cyclopropy-lmethoxy-quinazoline,
4-[(R)-(1-phenyl-ethyl)amino]-6-({4-[N-(2-methoxy-ethyl)-N-ethyl-amino]-1-oxo-2-buten-1-yl}amino)-7-cyclopropylmethoxy-quinazoline,
4-[(R)-(1-phenyl-ethyl)amino]-6-({4-[N-(2-methoxy-ethyl)-N-methyl-amino]-1-oxo-2-buten-1-yl}amino)-7-cyclopropylmethoxy-quinazoline,
4-[®-(1-phenyl-ethyl)amino]-6-({4-[N-(tetrahydropyran-4-yl)-N-methyl-amino]-1-oxo-2-buten-1-yl}amino)-7-cyclopropylmethoxy-quinazoline,
4-[(3-chloro-4-fluorophenyl)amino]-6-{[4-(N,N-dimethy-lamino)-1-oxo-2-buten-1-yl]amino}-7-((R)-tetrahydrofuran-3-yloxy)-quinazoline,
4-[(3-chloro-4-fluorophenyl)amino]-6-{[4-(N,N-dimethy-lamino)-1-oxo-2-buten-1-yl]amino}-7-((S)-tetrahydrofuran-3-yloxy)-quinazoline,
4-[(3-chloro-4-fluorophenyl)amino]-6-({4-[N-(2-methoxy-ethyl)-N-methyl-amino]-1-oxo-2-buten-1-yl}amino)-7-cyclopentyloxy-quinazoline,
4-[(3-chloro-4-fluorophenyl)amino]-6-{[4-(N-cyclopropyl-N-methyl-amino)-1-oxo-2-buten-1-yl]amino}-7-cyclo-pentyloxy-quinazoline,
4-[(3-chloro-4-fluorophenyl)amino]-6-{[4-(N,N-dimethy-lamino)-1-oxo-2-buten-1-yl]amino}-7-[(R)-(tetrahydrofuran-2-yl)methoxy]-quinazoline,
4-[(3-chloro-4-fluorophenyl)amino]-6-{[4-(N,N-dimethy-lamino)-1-oxo-2-buten-1-yl]amino}-7-[(S)-(tetrahydrofuran-2-yl)methoxy]-quinazoline,
4-[(3-ethynyl-phenyl)amino]-6,7-to-(2-methoxy-ethoxy)-quinazoline-[(3-chloro-4-fluorophenyl)amino]-7-[3-(morpholin-4-yl)-4propyloxy]-6-[(vinyl- carbonyl)amino]-quinazoline,
4-[(R)-(1-phenyl-ethyl)amino]-6-(4-hydroxy-phenyl)-7H-pyrrolo[2,3-d]pyrimidine
3-cyano-4-[(3-chloro-4-fluorophenyl)amino]-6-{[4-(N,N-dimethylamino)-1-oxo-2-buten-1-yl]amino}-7-ethoxy-quinoline,
4-{[3 -chloro-4-(3-fluoro-benzyloxy)-phenyl]amino}-6-(5-{[(2-methanesulphonyl-ethyl)amino]methyl}-furan-2-yl) quinazoline,
4-[(R)-(1-phenyl-ethyl)amino]-6-{[4-((R)-6-methyl-2-oxo-morpholin-4-yl)-1-oxo-2-buten-1-yl]amino}-7-methoxy-quinazoline,
4-[(3-chloro-4-fluorophenyl)amino]-6-{[4-(morpholin-4-yl)-1-oxo-2-buten-1-yl]-amino}-7-[(tetrahydrofuran-2-yl)methoxy]-quinazoline,
4-[(3-chloro-4-fluorophenyl)amino]-6-({4-[N,N-to-(2-methoxy-ethyl)-amino]-1-oxo-2-buten-1-yl}amino)-7-[(tetrahydrofuran-2-yl)methoxy]- quinazoline,
4-[(3-ethynylphenyl)amino]-6-{[4-(5,5 -dimethyl-2-oxo-morpholin-4-yl)-1-oxo-2-buten-1-yl]amino}-quinazoline,
4-[(3-chloro-4-fluorophenyl)amino]-6-[2-(2,2-dimethyl-6-oxo-morpholin-4-yl)-ethoxy]-7-methoxy-quinazoline,
4-[(3-chloro-4-fluorophenyl)amino]-6-[2-(2,2-dimethyl-6-oxo-morpholin-4-yl)-ethoxy]-7-[(R)-(tetrahydrofuran-2-yl)methoxy]-quinazoline,
4-[(3-chloro-4-fluorophenyl)amino]-7-[2-(2,2-dimethyl-6-oxo-morpholin-4-yl)-ethoxy]-6-[(S)-(tetrahydrofuran-2-yl)methoxy]-quinazoline,
4-[(3-chloro-4-fluorophenyl)amino]-6-{2-[4-(2-oxo-morpholin-4-yl)-piperidin-1-yl]-ethoxy}-7-methoxy-quinazoline,
4-[(3-chloro-4-fluorophenyl)amino]-6-[1-(tert.-butyloxy-carbonyl)-piperidin-4-yloxy]-7-methoxy-quinazoline,
4-[(3-chloro-4-fluorophenyl)amino]-6-(trans-4-amino-cyclohexan-1-yloxy)-7-methoxy-quinazoline,
4-[(3-chloro-4-fluorophenyl)amino]-6-(trans-4-methane-sulphonylamino-cyclohexan-1-yloxy)-7-methoxy-quinazoline,
4-[(3-chloro-4-fluorophenyl)amino]-6-(tetrahydropyran-3-yloxy)-7-methoxy-quinazoline,
4-[(3-chloro-4-fluorophenyl)amino]-6-(1-methyl-piperi-din-4-yloxy)-7-methoxy-quinazoline,
4-[(3-chloro-4-fluorophenyl)amino]-6-{1-[(morpholin-4-yl)carbonyl]-piperidin-4-yloxy}-7-methoxy-quinazoline,
4-[(3-chloro-4-fluorophenyl)amino]-6-{1-[(methoxym-ethyl)carbonyl]-piperidin-4-yloxy}-7-methoxy-quinazoline,
4-[(3-chloro-4-fluorophenyl)amino]-6-(piperidin-3-yloxy)-7-methoxy-quinazoline,
4-[(3-chloro-4-fluorophenyl)amino]-6-[1-(2-acetylaminoethyl)-piperidin-4-yloxy]-7-methoxy-quinazoline,
4-[(3-chloro-4-fluorophenyl)amino]-6-(tetrahydropyran-4-yloxy)-7-ethoxy-quinazoline,
4-[(3-chloro-4-fluorophenyl)amino]-6-((S)-tetrahydrofuran-3-yloxy)-7-hydroxy-quinazoline,
4-[(3-chloro-4-fluorophenyl)amino]-6-(tetrahydropyran-4-yloxy)-7-(2-methoxy-ethoxy)-quinazoline,
4-[(3-chloro-4-fluorophenyl)amino]-6-{trans-4-[(dimethylamino)sulphonylamino]-cyclohexan-1-yloxy}-7-methoxy-quinazoline,
4-[(3-chloro-4-fluorophenyl)amino]-6-{trans-4-[(morpholin-4-yl)carbonylamino]-cyclohexan-1-yloxy}-7-methoxy-quinazoline,
4-[(3-chloro-4-fluorophenyl)amino]-6-{trans-4-[(morpholin-4-yl)sulphonylamino]-cyclohexan-1-yloxy}-7-methoxy-quinazoline,
4-[(3-chloro-4-fluorophenyl)amino]-6-(tetrahydropyran-4-yloxy)-7-(2-acetylamino-ethoxy)-quinazoline,
4-[(3-chloro-4-fluorophenyl)amino]-6-(tetrahydropyran-4-yloxy)-7-(2-methanesulphonylamino-ethoxy)-quinazoline,
4-[(3-chloro-4-fluorophenyl)amino]-6-{1-[(piperidin-1-yl)carbonyl]-piperidin-4-yloxy}-7-methoxy-quinazoline,
4-[(3-chloro-4-fluorophenyl)amino]-6-(1-aminocarbonyl-methyl-piperidin-4-yloxy)-7-methoxy-quinazoline,
4-[(3-chloro-4-fluorophenyl)amino]-6-(cis-4-{N-[(tetrahydropyran-4-yl)carbonyl]-N-methyl-amino}-cyclohexan-1-yloxy)-7-methoxy-quinazoline,
4-[(3-chloro-4-fluoro-phenyl)amino]-6-(cis-4-{N-[(morpholin-4-yl)carbonyl]-N-methyl-amino}-cyclohexan-lyloxy)-7-methoxy-quinazoline,
4-[(3-chloro-4-fluorophenyl)amino]-6-(cis-4-{N-[(morpholin-4-yl)sulphonyl]-N-methyl-amino}-cyclohexan-1-yloxy)-7-methoxy-quinazoline,
4-[(3-chloro-4-fluorophenyl)amino]-6-(trans-4-ethane-sulphonylamino-cyclohexan-1-yloxy)-7-methoxy-quinazoline,
4-[(3-chloro-4-fluorophenyl)amino]-6-(1-methanesulphonyl-piperidin-4-yloxy)-7-ethoxy-quinazoline,
4-[(3-chloro-4-fluorophenyl)amino]-6-(1-methanesulphonyl-piperidin-4-yloxy)-7-(2-methoxy-ethoxy)-quinazoline,
4-[(3-chloro-4-fluorophenyl)amino]-6-[1-(2-methoxy-acetyl)-piperidin-4-yloxy]-7-(2-methoxy-ethoxy)-quinazoline,
4-[(3-chloro-4-fluorophenyl)amino]-6-(cis-4-acetylamino-cyclohexan-1-yloxy)-7-methoxy-quinazoline,
4-[(3-ethynyl-phenyl)amino]-6-[1-(tert.-butyloxycarbonyl)-piperidin-4-yloxy]-7-methoxy-quinazoline,
4-[(3-ethynyl-phenyl)amino]-6-(tetrahydropyran-4-yloxy]-7-methoxy-quinazoline,
4-[(3-chloro-4-fluoro-phenyl)amino]-6-(cis-4-{N-[(piperi-din-1-yl)carbonyl]-N-methyl-amino}-cyclohexan-1-yloxy)-7-methoxy-quinazoline,
4-[(3-chloro-4-fluorophenyl)amino]-6-(cis-4-{N-[(4-methyl-piperazin-1-yl)carbonyl]-N-methyl-amino}-cyclo-hexan-1-yloxy)-7-methoxy-quinazoline,
4-[(3-chloro-4-fluorophenyl)amino]-6-{cis-4-[(morpholin-4-yl)carbonylamino]-cyclohexan-1-yloxy}-7-methoxy-quinazoline,
4-[(3-chloro-4-fluorophenyl)amino]-6-{1-[2-(2-oxopyrro-lidin-1-yl)ethyl]-piperidin-4-yloxy}-7-methoxy-quinazoline,
4-[(3-chloro-4-fluorophenyl)amino]-6-{1-[(morpholin-4-yl)carbonyl]-piperidin-4-yloxy}-7-(2- methoxy-ethoxy)-quinazoline,
4-[(3-ethynyl-phenyl)amino]-6-(1-acetyl-piperidin-4-yloxy)-7-methoxy-quinazoline,
4-[(3-ethynyl-phenyl)amino]-6-(1-methyl-piperidin-4-yloxy)-7-methoxy-quinazoline,
4-[(3-ethynyl-phenyl)amino]-6-(1-methanesulphonyl-pip-eridin-4-yloxy)-7-methoxy-quinazoline,
4-[(3-chloro-4-fluoro-phenyl)amino]-6-(1-methyl-piperi-din-4-yloxy)-7(2-methoxy-ethoxy)-quinazoline,
4-[(3-chloro-4-fluorophenyl)amino]-6-(1-isopropyloxycar-bonyl-piperidin-4-yloxy)-7-methoxy-quinazoline,
4-[(3-chloro-4-fluorophenyl)amino]-6-(cis-4-methylamino-cyclohexan-1-yloxy)-7-methoxy-quinazoline,
4-[(3 -chloro-4-fluorophenyl)amino]-6-{cis-4-[N-(2-methoxy-acetyl)-N-methyl-amino]-cyclohexan-1-yloxy}-7-methoxy-quinazoline,
4-[(3-ethynyl-phenyl)amino]-6-(piperidin-4-yloxy)-7-methoxy-quinazoline,
4-[(3-ethynyl-phenyl)amino]-6-[1-(2-methoxy-acetyl)-piperidin-4-yloxy]-7-methoxy-quinazoline,
4-[(3-ethynyl-phenyl)amino]-6-{1-[(morpholin-4-yl)carbonyl]-piperidin-4-yloxy}-7-methoxy-quinazoline,
4-[(3-chloro-4-fluorophenyl)amino]-6-{1-[(cis-2,6-dimethyl-morpholin-4-yl)carbonyl]-piperidin-4-yloxy}-7-methoxy-quinazoline,
4-[(3 -chloro-4-fluorophenyl)amino]-6-{1-[(2-methyl-morpholin-4-yl)carbonyl]-piperidin-4-yloxy}-7-methoxy-quinazoline,
4-[(3-chloro-4-fluorophenyl)amino]-6-{1-[(S,S)-(2-oxa-5-aza-bicyclo[2,2,1]hept-5-yl)carbonyl]-piperidin-4-yloxy}-7-methoxy-quinazoline,
4-[(3-chloro-4-fluorophenyl)amino]-6-{1-[(N-methyl-N-2-methoxyethyl-amino)carbonyl]-piperidin-4-yloxy}-7-methoxy-quinazoline,
4-[(3-chloro-4-fluorophenyl)amino]-6-(1-ethyl-piperidin-4-yloxy)-7-methoxy-quinazoline,
4-[(3-chloro-4-fluorophenyl)amino]-6-{1-[(2-methoxy-ethyl)carbonyl]-piperidin-4-yloxy}-7-methoxy-quinazoline,
4-[(3-chloro-4-fluorophenyl)amino]-6-{1-[(3-methoxypropyl-amino)-carbonyl]-piperidin-4-yloxy}-7-methoxy-quinazoline,
4-[(3-chloro-4-fluorophenyl)amino]-6-[cis-4-(N-methane-sulphonyl-N-methyl-amino)-cyclohexan-1-yloxy]-7-methoxy-quinazoline,
4-[(3-chloro-4-fluorophenyl)amino]-6is-4-(N-acetyl-N-methyl-amino)-cyclohexan-1-yloxy]-7-methoxy-quinazoline,
4-[(3-chloro-4-fluorophenyl)amino]-6-(trans-4-methylamino-cyclohexan-1-yloxy)-7-methoxy-quinazoline,
4-[(3-chloro-4-fluorophenyl)amino]-6-[trans-4-(N-meth-anesulphonyl-N-methyl-amino)-cyclohexan-1-yloxy]-7-methoxy-quinazoline,
4-[(3-chloro-4-fluorophenyl)amino]-6-(trans-4-dimethylamino-cyclohexan-1-yloxy)-7-methoxy-quinazoline,
4-[(3-chloro-4-fluorophenyl)amino]-6-(trans-4-{N-[(morpholin-4-yl)carbonyl]-N-methyl-amino}-cyclohexan-1yloxy)-7-methoxy-quinazoline,
4-[(3-chloro-4-fluorophenyl)amino]-6-[2-(2,2-dimethyl-6-oxo-morpholin-4-yl)-ethoxy]-7-[(S)-(tetrahydrofuran-2-yl)methoxy]-quinazoline,
4-[(3-chloro-4-fluorophenyl)amino]-6-(1-methanesulphonyl-piperidin-4-yloxy)-7-methoxy-quinazoline,
and 4-[(3-chloro-4-fluorophenyl)amino]-6-(1-cyan-piperidin-4-yloxy)-7-methoxy-quinazoline.

The pharmaceutical active substances described above are optionally in the form of the racemates, enantiomers, diaste-reomers thereof and optionally in the form of the pharmacologically acceptable acid addition salts, solvates or hydrates thereof. According to the invention the acid addition salts of the betamimetics are preferably selected from among the hydrochloride, hydrobromide, hydriodide, hydrosulphate, hydrophosphate, hydromethanesulphonate, hydronitrate, hydromaleate, hydroacetate, hydrocitrate, hydrofumarate, hydrotartrate, hydroxalate, hydrosuccinate, hydrobenzoate and hydro-p-toluenesulphonate.

The dopamine agonists are preferably selected from among bromocriptin, cabergoline, alpha-dihy-droergocryptine, lisuride, pergolide, pramipexol, roxindol, ropinirol, talipexol, tergurid and viozan, optionally in the form of the racemates, enantiomers, diastereomers thereof and optionally in the form of the pharmacologically acceptable acid addition salts, solvates or hydrates thereof. According to the invention the acid addition salts of the betamimetics are preferably selected from among the hydrochloride, hydro-bromide, hydriodide, hydro sulphate, hydrophosphate, hydromethanesulphonate, hydronitrate, hydromaleate, hydroacetate, hydrocitrate, hydro fumarate, hydrotartrate, hydrooxalate, hydrosuccinate, hydrobenzoate and hydro-p-toluenesulphonate.

H1-Antihistamines which may be used are preferably compounds selected from among desloratadine, olopatadine, azelastine, epinastine, cetirizine, azelas-tine, fexofenadine, levocabastine, loratadine, mizolastine, ketotifen, emedastine, dimetindene, clemastine, bamipine, cexchlorpheniramine, pheniramine, doxylamine, chlorophe-noxamine, dimenhydrinate, diphenhydramine, promethazine, ebastine, desloratidine and meclozine, optionally in the form of the racemates, enantiomers, diastereomers thereof and optionally in the form of the pharmacologically acceptable acid addition salts, solvates or hydrates thereof. According to the invention the acid addition salts of the betamimetics are preferably selected from among the hydrochloride, hydro-bromide, hydriodide, hydrosulphate, hydrophosphate, hydromethanesulphonate, hydronitrate, hydromaleate, hydroacetate, hydrocitrate, hydrofumarate, hydrotartrate, hydroxalate, hydrosuccinate, hydrobenzoate and hydro-p-toluenesulphonate.

It is also possible to use inhalable macromolecules as disclosed in EP 1 003 478 A1 or CA 2297174 A1.

In addition, the compound may come from the groups of ergot alkaloid derivatives, the triptans, the CGRP-inhibitors, the phosphodiesterase-V inhibitors, interleukin-5 antagonists, kinase inhibitors, anti-PD-1, endothelin receptor antagonists, antibiotics, surfactants, anti-IgE, mast cell stabilizers, anti-RSV, CFTR-relevant compounds, IPF treatments, sGC stimulators, vasodilators, Alpha-Proteinase Inhibitors, antiarrhythmic, enzymes, sclerosing agents optionally in the form of the racemates, enantiomers or diastereomers thereof, optionally in the form of the pharmacologically acceptable acid addition salts, the solvates and/or hydrates thereof.

Examples of ergot alkaloid derivatives are dihydroergota-mine and ergotamine.

Examples of interleukin-5 antagonists are reslizumab and mepolizumab.

Examples of kinase inhibitors are alectinib, osimertinib, nintedanib, ceritinib, crizotinib and gefitinib.

Examples of anti-PD-1 are pembrolizumab and nivolumab.

Examples of endothelin receptor antagonists are macitentan, ambrisentan and bosentan.

Examples of antibiotics are telavancin, bedaquiline, aztreonam, ceftaroline, tigecycline, telithromycin, moxifloxacin, clarithromycin, ceftibuten, piperacillin, tazobactam, amoxicillin/clavulanate, sparfloxacin, grepafloxacin, cefdinir, ciprofloxacin, cefuroxime, dirithromycin, Gatifloxacin, ertapenem, Cefazolin, rifapentine and tobramycin.

Examples of surfactants are lucinactant, calfactant and poractant alpha.

Example of phosphodiesterase-V inhibitors is tadalafil.

Example of anti-IgE is omalizumab.

Examples of mast cell stabilizers are nedocromil and cromolyn.

Example of anti-RSV is palivizumab.

Examples of CFTR-relevant compounds are lumacaftor and ivacaftor.

Example of IPF-treatments is pirfenidone.

Example of sGC stimulators is riociguat.

Example of vasodilators is treprostinil.

Examples of antiarrhythmic are dofetilide and verapamil.

Examples of enzymes are dornase alfa and laronidase.

Example of sclerosing agents is sterile talc.

According to the invention, the most preferably pharmaceutical active ingredient is tiotropium bromide or tiotropium bromide monohydrate.

According to the invention, an inhalable powder pharmaceutical compositions containing tiotropium bromide, preferably in an amount of 0.001% to 5%, in admixture with a physiologically acceptable excipient is administered.

According to the invention, an inhalable powder containing 0.01% to 2%, preferably 0.04% to 0.8%, more preferably 0.08% to 0.64% tiotropium bromide in admixture with a physiologically acceptable excipient is administered.

More preferably according to the invention, an inhalable powder pharmaceutical compositions containing 0.12% to 0.48% tiotropium bromide in admixture with a physiologically acceptable excipient is administered.

More preferably according to the invention, an inhalable powder pharmaceutical compositions containing 0.12% to 0.5% crystalline tiotropium bromide monohydrate in admixture with a physiologically acceptable excipient is administered.

According to the invention, physiologically acceptable excipients used to prepare the inhalable powders applicable may include monosaccharides (e.g., glucose or arabinose), disaccharides (e.g., lactose, saccharose, or maltose), oligo- and polysaccharides (e.g., dextrane), polyalcohols (e.g., sorbitol, mannitol, or xylitol), salts (e.g., sodium chloride or calcium carbonate) or mixtures of these excipients with one another. Mono- or disaccharides, particularly, lactose or glucose in the form of their monohydrates, are preferably used for excipient.

### EXAMPLES

The following examples are given to illustrate the present invention. It should be understood, however, that the spirit and scope of the invention is not to be limited to the specific conditions or details described in these examples.

### Example 1

### 1.1. Excipient Mixture

8.8 kg of course lactose monohydrate with an average particle size of 80 μm is used as the coarser excipient component. 1.2 kg of lactose monohydrate with an average particle size of 6 μm is used as the finer excipient component. In the resulting 10.0 kg of excipient mixture the proportion of the finer excipient component is 12%.

About 4.4 kg of course lactose monohydrate is added to a suitable mixing container. Then, about 1.2 kg finer lactose monohydrate is added. Remaining course lactose monohydrate for inhalation is finally added. The ingredients are then mixed together.

### 1.2. Final Mixture:

To prepare the final mixture, 10 kg of the excipient mixture and 0.041 kg of micronized tiotropium bromide monohydrate with an average particle size of 3.2 μm are used. The content of active substance in the resulting 10.041 kg of inhalable powder is 0.4%.

About 1.0 to 2.0 kg of excipient mixture is added to a suitable mixing container. Alternate layers of tiotropium bromide monohydrate are about 0.01 kg. The excipient mixture and the active substance are added in layers, respectively. The ingredients are then mixed together.

Those embodiments stated in this invention are only used as illustrations of the principles and applications of the present invention. Consequently it ought to be aware that other modifications may be applicable to the illustrative embodiments without the departure of the spirit and realm of the present invention as defined by the claims appended.

### LIST OF REFERENCE NUMERALS

1: Cap
2: Mouthpiece
3: Deck

1. An inhaler device for inhaling powdered pharmaceutical compositions from capsules comprising a base, a deck, a chamber, a mouthpiece and a cap, wherein the deck can be latched to the base and closed off, and being connected to a push button through a protrusion and a sliding groove, and wherein the capsule-piercing unit comprising the push button, the spring fixture, two piercing pins and a spring, and wherein the push button and spring fixture are not hinged together with two separated parts, and wherein the base, the deck, the mouthpiece and the cap are hinged together by a single joint spindle.

2. An inhaler device for inhaling powdered pharmaceutical compositions from capsules comprising a base, a deck, a chamber, a mouthpiece and a cap, wherein the deck can be latched to the base and closed off, and being connected to a push button through a protrusion and a sliding groove, and wherein the capsule-piercing unit comprising the push button, the spring fixture, two piercing pins and a spring, and wherein the push button and spring fixture are not hinged together with two separated parts, and wherein the cap is not hinged together by a single spindle joint with the rest of the device and instead is held together with the rest of the inhaler device by means of a bugle on the inside of the cap along with the corresponding dent on the mouthpiece, and by means of the protective shell with a strip pattern.

3. An inhaler device for inhaling powdered pharmaceutical compositions from capsules comprising a base, a deck, a chamber, a mouthpiece and a cap, wherein the deck can be latched to the base and closed off, and being connected to a push button through a protrusion and a sliding groove, and wherein the capsule-piercing unit comprising the push button, the spring fixture, two piercing pins and a spring, and wherein the push button and spring fixture are not hinged together with two separated parts, and wherein the cap is hinged to the rest of the device by means of a linking belt which attaches the cap and the push button on both sides.

4. The inhaler device according to claims 1, 2 or 3, wherein the deck has a pair of buckles near push button to ensure tight connection between the deck and the base.

5. The inhaler device according to claims 1, 2 or 3, wherein the spring fixture has two arms. Each arm has at least one bulge to ensure the arm could be entrapped in the each side of the chamber.

6. The inhaler device according to claims 1, 2 or 3, wherein the push button has a protrusion.

7. The inhaler device according to claims 1, 2 or 3, wherein the protrusion is entrapped in the sliding groove and is movable in the sliding groove.

8. The inhaler device according to claim 1, wherein the inhaler device is for inhalation of powdered pharmaceutical compositions capsules.

9. The inhaler device according to claim 2, wherein the inhaler device is for inhalation of powdered pharmaceutical compositions from capsules.

10. The inhaler device according to the claim 3, wherein the inhaler device is for inhalation of powdered pharmaceutical compositions from capsules.

11. The powdered pharmaceutical compositions according to the claims 8, 9 or 10, wherein the powdered pharmaceutical compositions comprising pharmaceutical active substance in admixture with a physiologically acceptable excipient.

12. The pharmaceutical active substance according to the claim 11, wherein the pharmaceutical active substance is selected from anticholinergics, betamimetics, steroids, phosphodiesterase IV-inhibitors, LTD4-antagonists and EGFR-kinase-inhibitors, antiallergics, ergot alkaloids derivatives, triptans, CGRP-antagonists, phosphodiesterase-V-inhibitors and any combination of each of the foregoing.

13. The pharmaceutical active substance according to the claim 11, wherein the pharmaceutical active substance is selected from tiotropium, tiotropium bromide and tiotropium bromide monohydrate.

14. The physiologically acceptable excipient according to the claim 11, wherein the physiologically acceptable excipient is selected from lactose, lactose hydrates and lactose monohydrate.
